(19) United States
(12) Patent Application Publication
(10) Pub. No.: US 2016/0367385 A1
(43) Pub. Date: Dec. 22, 2016

(54) PROSTHETIC KNEE JOINT AND CONTROL METHOD THEREOF

(71) Applicant: NABTESCO CORPORATION, Tokyo (JP)

(72) Inventors: Hiroaki HASHIMOTO, Kobe-shi (JP); Yoshiaki NAKAYA, Kobe-shi (JP); Masakazu SAITO, Kobe-shi (JP); Yoshihiro TADA, Kobe-shi (JP); Taishiro MISAO, Kobe-shi (JP); Masahiko OKUDA, Kobe-shi (JP)

(21) Appl. No.: 15/183,338

(22) Filed: Jun. 15, 2016

(30) Foreign Application Priority Data
Jun. 19, 2015 (JP) 2015-124176

(51) Int. Cl. A61F 2/68; A61F 2/64

(52) U.S. Cl. CPC A61F 2/68; A61F 2/64; A61F 2002/704

(57) ABSTRACT

A prosthetic knee joint includes a knee portion coupled to the socket, a cylinder that is coupled to the knee portion and limits or aids operation of the knee portion, a knee angle sensor that directly or indirectly detects a knee angle value, and a control unit that controls driving of the cylinder. The control unit may calculate a knee angular velocity value based on the knee angle value detected by the knee angle sensor. The control unit determines whether the knee portion is in a knee still state based on the knee angular velocity value and automatically restricts contraction of the cylinder based on a time duration in which the knee portion is in the knee still state.

### CROSS-REFERENCE TO RELATED APPLICATIONS

This application is based on and claims the benefit of priority from Japanese Patent Application Serial No. 2015-124176 filed on Jun. 19, 2015, the contents of which are hereby incorporated by reference in their entirety.

### TECHNICAL FIELD

The present invention relates to a prosthetic knee joint and a control method thereof.

### BACKGROUND

A typical prosthetic knee joint includes an upper portion that supports a socket at its upper end, a lower portion that supports a foot portion at its lower end, a knee joint body that flexurally couples the upper portion and the lower portion, and a cylinder device that aids or restricts the flexural movements. The cylinder device contracts and the length of the cylinder becomes small when the knee flexes, whereas the cylinder device extends and the length of the cylinder becomes large when the knee is straighten. The cylinder device allows the knee flexion and extension.

Patent Literature 1 discloses a conventional prosthetic knee joint. The prosthetic knee joint disclosed in Patent Literature 1 includes a plurality of sensors. Moreover the knee joint is configured to change a flexion resistance and/or extension resistance of an actuator based on data provided from the plurality of sensors. However since such a prosthetic knee joint uses many sensors, the control is complicated and the cost tends to be increased. Furthermore, since the system used in the prosthetic knee joint is complicated, there is a possibility of decreased reliability.

### RELEVANT REFERENCES

### List of Relevant Patent Literature

Patent Literature 1: Japanese Patent Application Publication No. 2013-510604

### SUMMARY

Meanwhile, a user of a prosthetic leg that includes the prosthetic knee joint may try to pause in the posture where the user stoops down and bends his/her knees slightly. At this point, the user may be able to bent his/her knees at a desired angle for a moment to hold the prosthetic knee joint still by using an unimpaired leg to support his/her weight and extending his/her hip joints. However staying in such a posture for a long time may impose a great burden to the user of the prosthetic leg.

In view of the above, one object of the invention is to provide a prosthetic knee joint and a control method thereof in which contraction of a cylinder can be restricted with a simple configuration in accordance with an intention of a user of the prosthetic leg who tries to stay still in a posture where the user stoops down and bends his/her knees slightly.

### SUMMARY

A prosthetic knee joint according to an aspect of the invention couples a foot portion and a socket that corresponds to a thigh of a prosthesis user. The prosthetic knee joint includes: a knee portion coupled to the socket; a cylinder that is coupled to the knee portion and limits or aids operation of the knee portion; a knee angle sensor that directly or indirectly detects a knee angle value which is an angle of the knee portion; and a control unit that is coupled to the knee angle sensor and controls driving of the cylinder. The control unit calculates a knee angular velocity value based on the knee angle value detected by the knee angle sensor, and the control unit determines whether the knee portion is in a knee still state based on the knee angular velocity value and automatically restricts contraction of the cylinder based on a time duration in which the knee portion is in the knee still state.

In the above prosthetic knee joint, the control unit may restrict contraction of the cylinder when the knee angle value is on a knee flexion side with reference to a predetermined value.

The above prosthetic knee joint may further include a load sensor directly or indirectly detecting a load value of the prosthetic knee joint on the foot portion. The control unit may restrict contraction of the cylinder when the load value provided from the load sensor is larger than a predetermined value.

In the above prosthetic knee joint, the control unit may lift the restriction on contraction of the cylinder when the load value exceeds an upper-limit load threshold value that is larger than the predetermined value.

In the above prosthetic knee joint, the control unit may automatically restricts contraction of the cylinder when the knee still state continues for a predetermined time duration T₁.

In the above prosthetic knee joint, after contraction of the cylinder is restricted, the control unit may automatically lift the restriction on contraction of the cylinder when the load value is below a predetermined value or when the knee angle value changes to a value obtained at a time of knee extension and the knee angle value continues to be below a predetermined threshold value for a predetermined time duration T₂ or longer.

In the above prosthetic knee joint, after contraction of the cylinder is restricted, the control unit may automatically lift the restriction on contraction of the cylinder when the load value is below a predetermined value or when the knee angle changes to a value obtained at a time of knee extension and the knee angle value continues to be below a predetermined threshold value for a predetermined time duration T₂ or longer, and wherein relationship between the time duration T₁ and the time duration T₂ is represented as T₁>T₂.

In the above prosthetic knee joint, after contraction of the cylinder is restricted, the control unit may automatically lift the restriction on contraction of the cylinder when a rotational moment of the prosthetic knee joint on the socket in an extension direction exceeds a predetermined moment threshold value.

In the above prosthetic knee joint, the control unit may automatically restrict contraction of the cylinder when the prosthetic knee joint has experienced two states: one is that the knee angle value exceeds a predetermined knee angle change upper-limit threshold value and the other is that the knee angle value falls below a predetermined knee-angle change lower-limit threshold value.

A prosthetic knee joint according to another aspect of the invention couples a foot portion and a socket that corresponds to a thigh of a prosthesis user. The prosthetic knee joint includes: a knee portion coupled to the socket; a cylinder coupled to the knee portion, the cylinder that limits or aids operation of the knee portion; a knee angular velocity sensor that directly or indirectly detects a knee angular velocity value which is a change of angle of the knee portion per unit time; and control unit coupled to the knee angle sensor, the control unit that controls driving of the cylinder. The control unit determines whether the knee portion is in a knee still state based on the knee angular velocity value detected by the knee angular velocity sensor and automatically restricts contraction of the cylinder based on a time duration in which the knee portion is in the knee still state.

A prosthetic knee joint according to another aspect of the invention couples a foot portion and a socket that corresponds to a thigh of a prosthesis user. The prosthetic knee joint includes: a knee portion coupled to the socket; a cylinder coupled to the knee portion, the cylinder limits or aids operation of the knee portion; a knee angle sensor that directly or indirectly detects a knee angle value which is an angle of the knee portion; a control unit that is coupled to the knee angle sensor and controls driving of the cylinder; and a load sensor directly or indirectly detecting a load value of the prosthetic knee joint on the foot portion. After contraction of the cylinder is restricted, the control unit automatically lifts the restriction on contraction of the cylinder when the load value obtained from the load sensor is below a predetermined value or when the knee angle value shifts to a knee extension side and the shifting of the knee angle value continues for a predetermined time duration or longer.

According to another aspect of the invention, provided is a method of controlling a prosthetic knee joint that couples a foot portion and a socket that corresponds to a thigh of a prosthesis user. The prosthetic knee joint includes: a knee portion coupled to the socket; a cylinder coupled to the knee portion, the cylinder that limits or aids operation of the knee portion; and a knee angle sensor that directly or indirectly detects a knee angle value which is an angle of the knee portion. The method includes calculating a knee angular velocity value based on the knee angle value detected by the knee angle sensor, and determining whether the knee portion is in a knee still state based on the knee angular velocity value and automatically restricting contraction of the cylinder based on a time duration in which the knee portion is in the knee still state.

According to the aspects of the invention, it is possible to restrict contraction of the cylinder in accordance with an intention of a user of the prosthesis who tries to stoop down slightly, flex his/her knees a little and stay still in this posture.

### BRIEF DESCRIPTION OF THE DRAWINGS

FIG. 1 is an elevation view of an above-knee prosthesis that includes a prosthetic knee joint according to one embodiment of the invention.

FIG. 2 is a schematic view of the above-knee prosthesis that includes the prosthetic knee joint according to the embodiment of the invention.

FIG. 3 is a hydraulic circuit diagram including a cylinder and a drive mechanism.

FIG. 4a is a hydraulic circuit diagram including the cylinder and the drive mechanism at the time of knee flexion and FIG. 4b is a hydraulic circuit diagram including the cylinder and the drive mechanism at the time of knee extension.

### DESCRIPTION OF THE EMBODIMENTS

The embodiments of the invention will now be described with reference to the drawings.

Structure of Above-Knee Prosthesis

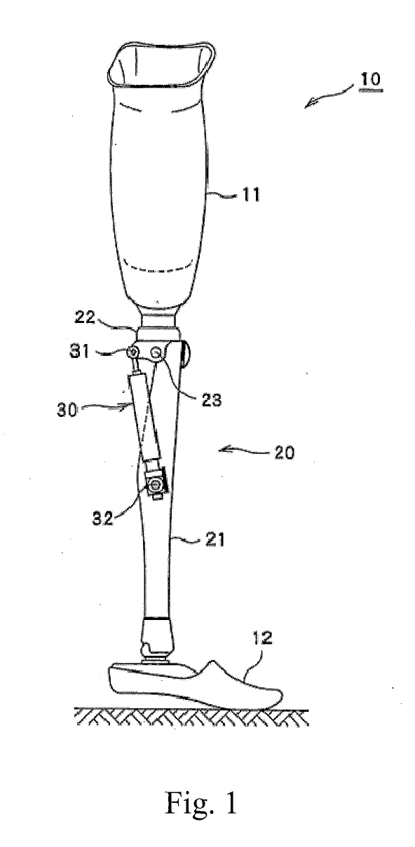

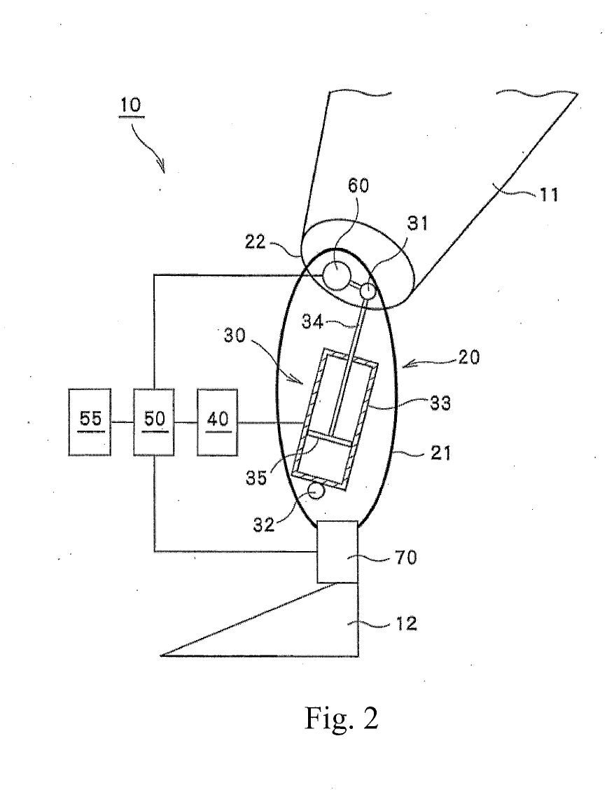

FIG. 1 is an elevation view of an above-knee prosthesis that includes a prosthetic knee joint according to the embodiment, and FIG. 2 is a schematic view of the above-knee prosthesis that includes the prosthetic knee joint.

Referring to FIG. 1, an above-knee prosthesis 10 may include a socket 11 that corresponds to a thigh and is made of plastic, a prosthetic knee joint 20 that corresponds to a lower leg and is rotatably coupled to the lower end of the socket, and a foot portion 12 that is coupled to the lower end of the prosthetic knee joint 20.

Structure of Prosthetic Knee Joint

Referring to FIG. 2, the prosthetic knee joint 20 may include a frame 21, a knee portion 22 that is configured to be rotatable with respect to the frame 21 and connected to the socket 11, a cylinder 30 that is coupled to the knee portion 22 and restricts or aids a rotational movement of the knee portion 22, and a drive mechanism 40 that drives the cylinder 30.

On the periphery of the cylinder 30, provided is a knee angle sensor 60 that detects a value of a knee angle which is an angle of the knee portion 22 based on an amount of contraction and extension of the cylinder 30. The knee angle sensor 60 may be connected to a control unit 50. Here the knee angle is an angle of the axis of the prosthetic knee joint 20 with the axis of the socket 11. When an user of the prosthetic leg stays upright, for example, the knee angle is 0° and the axis of the prosthetic knee joint 20 aligns with the axis of the socket 11 in the same line. Whereas when the user of the prosthetic leg is seated, for example, the knee angle becomes 90° and the axis of the prosthetic knee joint 20 becomes orthogonal to the axis of the socket 11. At the lower end of the frame 21, a load sensor 70 may be provided to detect a load value (vertical load) of the prosthetic knee joint 20 on the foot portion 12. The load sensor 70 may be connected to the control unit 50.

The control unit 50 may control operation of the drive mechanism 40 based on signals provided by the knee angle sensor 60 and the load sensor 70 in order to control driving of the cylinder 30. A battery 55 that provides power to various components including the drive mechanism 40 and the control unit 50 may be coupled to the control unit 50.

Although the drive mechanism 40, the control unit 50 and the battery 55 are shown outside the frame 21 in FIG. 2, they are actually attached to the frame 21 or the cylinder 30.

The cylinder 30 (a hydraulic cylinder) may use lubricant as a working fluid and may aid or restrict movement of the knee portion 22 by generating a resistive force. The cylinder 30 may have an upper supported point 31 situated close to a support point 23 (see FIG. 1) that couples the socket 11 and the frame 21 rotatably to each other, and a lower supported point 32 that is coupled to a portion of the frame 21. In this configuration, the cylinder 30 contracts and the length of the cylinder becomes small when the knee flexes, whereas the cylinder 30 extends and the length of the cylinder becomes large when the knee extends. Here the length of the cylinder 30 refers to the length between the upper supported point 31 and the lower supported point 32 of the cylinder 30.

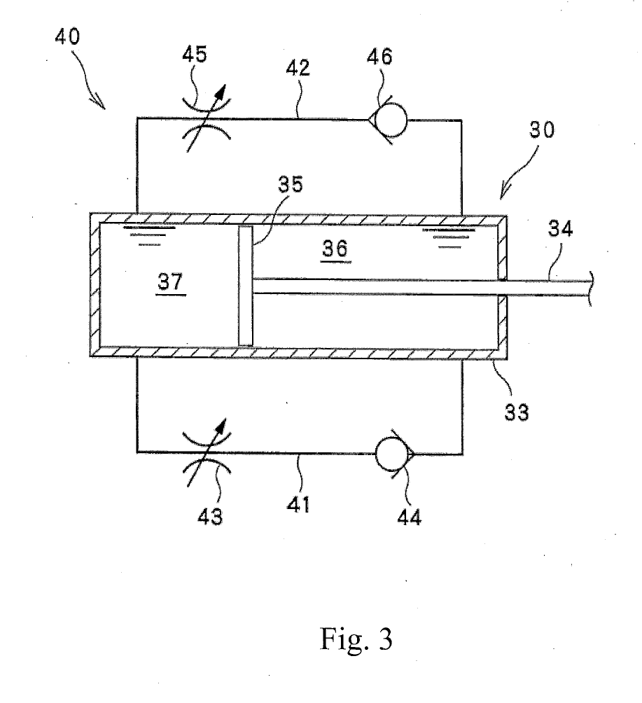

Operations of the cylinder 30 and the drive mechanism 40 will be now described with reference to FIG. 3. The cylinder 30 may include a cylinder tube 33, a piston rod 34 that is movable with respect to the cylinder tube 33, and a piston 35 that is shiftably housed within the cylinder tube 33 and to which the piston rod 34 is fixed. The internal space of the cylinder tube 33 may be divided into a first cavity 36 and a second cavity 37 by the piston 35. The first cavity 36 and the second cavity 37 may be filled with lubricant which is a working fluid.

The drive mechanism 40 is a mechanism for controlling the drive of the cylinder 30 utilizing a hydraulic pressure. The drive mechanism 40 may include an extension-side hydraulic circuit 41 and a flexion-side hydraulic circuit 42 that are coupled to the cylinder 30. The extension-side hydraulic circuit 41 and the flexion-side hydraulic circuit 42 are both communicated with the first cavity 36 and the second cavity 37 of the cylinder 30. The extension-side hydraulic circuit 41 may include an extension-side valve 43 and an extension-side check valve 44. The extension-side hydraulic circuit 41 can lead the lubricant from the first cavity 36 to the second cavity 37 but cannot lead the lubricant from the second cavity 37 to the first cavity 36. The flexion-side hydraulic circuit 42 may include a flexion-side valve 45 and a flexion-side check valve 46. The flexion-side hydraulic circuit 42 can lead the lubricant from the second cavity 37 to the first cavity 36 but cannot lead the lubricant from the first cavity 36 to the second cavity 37. The extension-side valve 43 and the flexion-side valve 45 may be coupled to the control unit 50 and configured to be fully-opened, fully-closed or in an intermediate state (partially opened) in response to the control of the control unit 50.

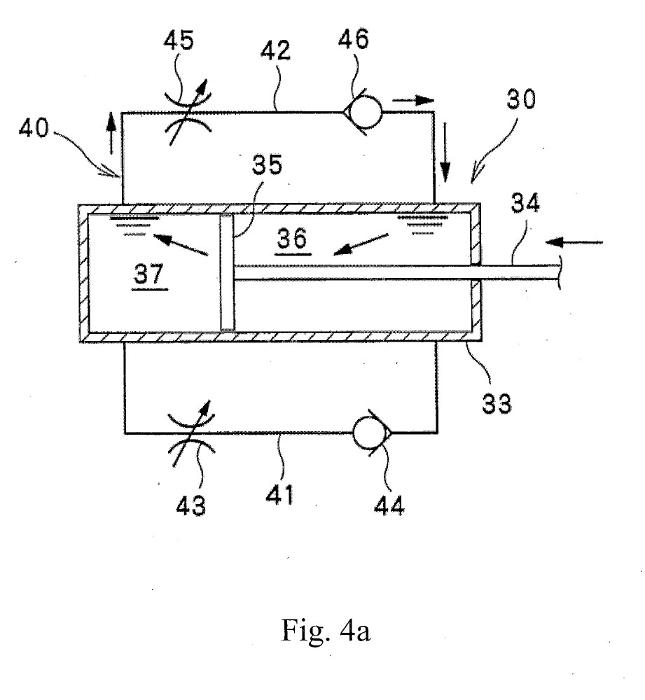

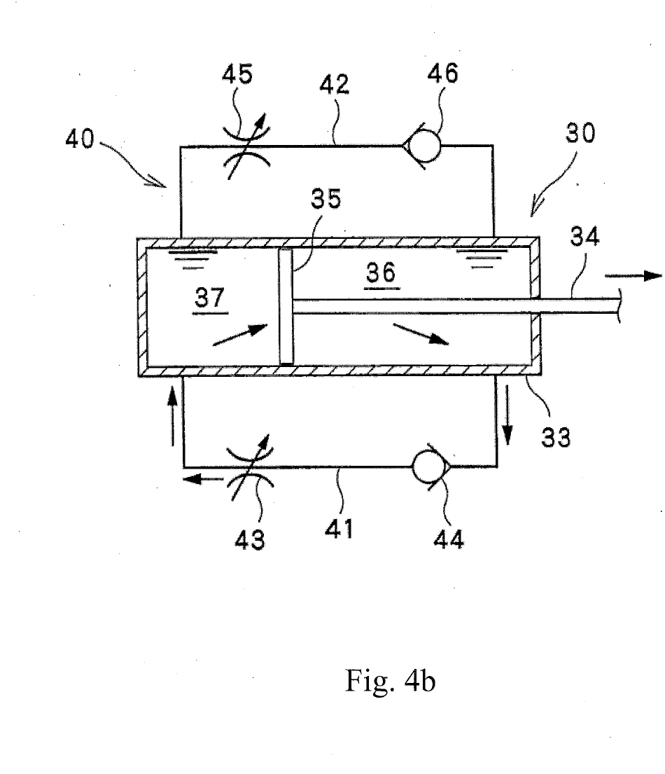

Referring to FIG. 4a, when the knee is bent (knee flexion), the piston rod 34 is contracted and the piston 35 moves to a retraction side. In this way, the lubricant from the second cavity 37 flows to the flexion-side hydraulic circuit 42 through the flexion-side valve 45 and then flows into the first cavity 36 through the flexion-side check valve 46. At this point, it is possible to restrict the contraction of the piston rod 34 by stopping the flow of the lubricant through the flexion-side hydraulic circuit 42 by closing the flexion-side valve 45. In this case, the movement of the cylinder 30 in the direction where the knee flexes is restricted.

Referring to FIG. 4b, when the knee is extended (knee extension), the piston rod 34 is extended and the piston 35 moves to a push-out side. In this way, the lubricant from the first cavity 36 flows to the extension-side hydraulic circuit 41 through the flexion-side check valve 44 and then flows into the second cavity 37 through the flexion-side valve 43. At this point, it is possible to restrict the extension of the piston rod 34 by stopping the flow of the lubricant through the extension-side hydraulic circuit 41 by closing the extension-side valve 43. In this case, the movement of the cylinder 30 in the direction where the knee extends is restricted.

Referring again to FIG. 2, the knee angle sensor 60 may include, for example, magnet (not shown) housed in the piston rod 34, and a magnetic sensor (not shown) that is fixed to the cylinder tube 33 and detects a position of the magnet. The knee angle sensor 60 may detect a knee angle based on a contracted or extended position of the piston rod 34 and may transmit the detected value of the knee angle to the control unit 50. The knee angle sensor 60 may not be limited to the one that directly detects the knee angle but may be ones that detect the knee angle indirectly. For example, the control unit 50 may perform numerical conversion of the contracted or extended position of the piston rod 34 that has been detected by the knee angle sensor 60 to obtain the knee angle value.

The load sensor 70 may be a strain sensor that is disposed between the socket 11 and the foot portion 12. In this case, the load sensor 70 may be provided to the socket 11 and may include a sensor block (not shown) and a strain sensor (not shown) attached to the sensor block. The load sensor 70 may detect a load value of the prosthetic knee joint 20 applied on the foot portion 12 by sensing a strain caused in the sensor block by the strain sensor. The load sensor 70 may transmit the load value to the control unit 50. The load value may not be directly detected but may be indirectly detected. For example, the control unit 50 may perform numerical conversion of the strain value detected by the load sensor 70 to obtain the load value.

The control unit 50 (computer) may be, for example, a Micro Control Unit (MCU). The control unit 50 may calculate a knee angular velocity based on the knee angle value detected by the knee angle sensor 60. The control unit 50 may control operation of the drive mechanism 40 based on signals provided by the knee angle sensor 60 and the load sensor 70 in order to control driving of the cylinder 30.

More specifically, the control unit 50 may calculate a knee angular velocity based on the knee angle value of the knee portion 22 detected by the knee angle sensor 60. The knee angular velocity is a change of the knee angle over time and refers to an amount of knee angle change per unit of time. In this case, the control unit 50 may determine whether the knee portion 22 remains still or not based on the angular velocity value obtained in the above-described way, the knee angle value obtained from the knee angle sensor 60, and the load value obtained from the load sensor 70. Here, unlike a normal gait state, the knee in the still state refers to a state where a user of the prosthetic leg tries to stay still in a posture in which the user stoops slightly down and flexes his/her knee a little, in other words, the state where the prosthetic knee joint 20 is slightly flexed with respect to the thigh.

The control unit 50 may automatically restrict (safety-lock) the contraction of the cylinder 30 based on a time duration in which the knee stays still, for instance, when the knee portion 22 is in the still state for a predetermined amount of time. More specifically, when a user of the prosthetic leg holds his/her knee still for a predetermined amount of time, it is determined that the user intends to get the prosthetic knee joint 20 still with respect to the thigh, and the contraction of the cylinder 30 is restricted. At this point, the displacement of the piston rod 34 with respect to the cylinder tube 33 is restricted. In this way, an angle of the knee 22 is fixed and thereby the prosthetic knee joint 20 can be fixed to the socket 11 (the thigh). As a result, the user of the prosthetic leg is able to keep the posture in which the user stoops slightly down and his/her knee is flexed a little without fatigue.

### Operation in the Embodiment

Operation of the control unit 50 to restrict the contraction of the cylinder 30 (a control method of the prosthetic knee joint 20) will be now described.

The knee angle sensor 60 may firstly detect an angle of the knee portion 22 continuously or periodically and transmit the detection result as a signal to the control unit 50. The control unit 50 may then calculate a knee angular velocity of the knee portion 22 based on the knee angle value detected by the knee angle sensor 60. In this case, the control unit 50 may obtain the knee angular velocity by, for example, differentiating the knee angle value. The load sensor 70 may detect a load (vertical load) of the prosthetic knee joint 20 applied on the foot portion 12 continuously or periodically and transmit the detection result as a signal to the control unit 50. The control unit 50 may store the knee angular velocity values, the knee angle values, and the load values in time-sequential manner.

Now when a user of the prosthetic leg stoops slightly and flexes his/her knee a little to remain still is considered. At this point, the knee angle value becomes substantially constant so that a change of the knee angular velocity becomes smaller than a knee angular velocity at the time when the user walks normally. A condition where the knee angular velocity value is equal to or smaller than a predetermined value (threshold value A) both in the extension direction and the flexion direction is referred to as condition A. For example, the condition A may be set such that the knee angular velocity value is equal to or smaller than 1°/50 ms.

When a user of the prosthetic leg stoops down slightly, flexes his/her knee a little and remains still, an angle of the knee portion 22 (an angle of the axis of the prosthetic knee joint 20 with axis of the socket 11) becomes non-zero and reaches to or over a predetermined value, moreover the knee angle value may be a value on a knee flexion side with reference to the predetermined value. A condition where the knee angle value is equal to or above the predetermined value (threshold value B) is referred to as condition B. For example, the condition B may be set such that the knee angle value is equal to or larger than 15°.

Moreover when a user of the prosthetic leg stoops down slightly, flexes his/her knees a little and remains still, a load of the prosthetic knee joint 20 is applied on the foot portion 12 in a vertical direction so that the load reaches to or over a predetermined value. A condition where the load value is equal to or above the predetermined value (lower-limit load threshold value C) is referred to as condition C. For example, the condition C may be set such that the load value is equal to or larger than 100 N.

When all the conditions A, B, and C are satisfied, the control unit 50 may determine that the knee portion 22 is in the knee still state. When the knee still state continues for a predetermined time duration T₁ or longer, the control unit 50 may restrict the contraction of the cylinder 30 automatically. The predetermined time duration T₁ may be set to any adequate value, for example, 1 to 3 seconds.

At this point, the control unit 50 may control the drive mechanism 40 to close the flexion-side valve 45. Consequently the lubricant does not flow through the flexion-side hydraulic circuit 42 and the contraction of the cylinder 30 is automatically restricted to the knee flexion side. Even when the user of the prosthetic leg puts a pressure on the knee portion, the angle of the knee portion 22 is not increased anymore so that the knee will not be flexed any more by the weight of the user. Therefore the user can easily maintain the knee still state and can easily hold the posture in which the user stoops slightly and flexes his/her knees a little.

At the same time, the control unit 50 may control the drive mechanism 40 to keep the extension-side valve 43 open. Accordingly the movement of the cylinder 30 is not restricted in the direction where the knee extends. Therefore the user of the prosthetic leg is able to quit the knee still state by, for example, extending the knee in accordance with his/her intention to lift the restriction on contraction of the cylinder 30.

After the contraction of the cylinder 30 is restricted, operation to lift the restriction on contraction of the cylinder 30 will be now described.

For example, considering a case where a user of the prosthetic leg tries to change his/her posture from the stoop to upright and to release the knee still state by extending the knee. In this case, the knee angle becomes smaller in the extension direction so that the change of the knee angular velocity becomes larger than that of the knee still state. More specifically, the knee angular velocity in the extension direction exceeds the predetermined value (the threshold value A). Consequently the condition A is dissatisfied. More specifically, for the above-example, the condition A may be dissatisfied when the knee angular velocity exceeds 1°/50 ms.

Moreover the angle of the knee portion 22 becomes smaller when the user changes his/her posture from the stoop to the upright, so that the knee angle value shifts to the knee extension side and becomes smaller than the predetermined value (the threshold value B). Consequently the condition B is dissatisfied. More specifically, for the above-example, the condition B may be dissatisfied when the knee angle becomes below 15°.

Alternatively the user of the prosthetic leg may release the knee still state by lifting the prosthesis up from the ground by putting his/her weight on the unimpaired leg or by sitting on a chair. In this case, a load of the prosthetic knee joint 20 on the foot portion 12 in the vertical direction is decreased so that the load value becomes below the predetermined value (the threshold value C) and the condition C is dissatisfied. More specifically, for the above-example, the condition C may be dissatisfied when the load value becomes below 100 N.

When at least one of the conditions A, B and C is dissatisfied, the control unit 50 may determine that the knee portion 22 is no longer in the still state. When the knee still state does not continue for a predetermined time duration T₂ or longer, the control unit 50 may automatically lift the restriction on contraction of the cylinder 30. For example, the control unit 50 may lift the restriction on contraction of the cylinder 30 when the load value does not reach to the predetermined value (the load value is below the threshold value C), or when the shift of the knee angle toward the knee extension side the knee angle value is below the threshold value B) continues for the predetermined time duration T₂ or longer. The predetermined time duration T₂ may be set to any adequate value, for example, 0.1 to 0.3 seconds.

It is preferable that the relationship between the time T₁ required to restrict the contraction of the cylinder 30 and the time T₂ required to lift the restriction on contraction of the cylinder 30 be T₁>T₂. More specifically, by setting T₁ to or above a predetermined value, it is possible to securely prevent the contraction of the cylinder 30 from being restricted at a normal operation such as when the user of the above-knee prosthesis walks down stairs, and thereby it is possible to enhance the safety. Whereas when T₂ is set to or below a predetermined value, the user is allowed to release the restriction on contraction of the cylinder 30 without waiting more than enough time, and thereby it is possible to increase the comfort at the time of use. Therefore, by satisfying the relationship T₁>T₂, it is possible to realize both the safety and the comfort.

When the restriction on contraction of the cylinder 30 is lifted, the control unit 50 may control the drive mechanism 40 to open the flexion-side valve 45. Consequently the lubricant stored in the second cavity 37 is allowed to flow into the flexion-side hydraulic circuit 42 and thereby the piston rod 34 of the cylinder 30 is smoothly extended. As a result, a user of the prosthetic leg can release the knee still state and get back to the normal operation smoothly.

Although the condition A (the knee angular velocity value), the condition B (the knee angle value), and the condition C (the load value) have been described as the conditions to restrict the contraction of the cylinder 30 by the control unit 50, all of the conditions may not be necessarily satisfied. For example, the contraction of the cylinder 30 may be restricted when only the condition A is satisfied.

However in the case where the contraction of the cylinder 30 is restricted when both the condition A and the condition B are satisfied, it is possible to prevent unnecessary restriction on contraction of the cylinder 30 when the user extends his/her legs to stand upright. Moreover, by setting the threshold value B larger than a threshold value of a knee angle (about 10°) for transition from a stance phase to a swing phase, it is possible to prevent the transition from the stance phase to the swing phase from being hampered due to unnecessary restriction on contraction of the cylinder 30.

Moreover in the case where the contraction of the cylinder 30 is restricted when both the condition A and the condition C are satisfied, it is possible to prevent unnecessary restriction on contraction of the cylinder 30, for example, when the user sits or descends stairs. Furthermore, the restriction on contraction of the cylinder 30 may be lifted even when the user of the prosthetic leg is not able to extend the knee like when the user sits in a car.

In addition to the condition A (the knee angular velocity value), the condition B (the knee angle value), and the condition C (the load value), alternatively the contraction of the cylinder 30 may be restricted or the restriction on contraction of the cylinder 30 may be lifted when at least one of the following conditions (condition D, condition E, and condition F) is satisfied.

The example in which the control unit 50 determines that the knee still state occurs when the load value exceeds the predetermined value (the threshold value C) (the condition C) and determines that the knee still state is not occurring when the load value is below the predetermined value (the threshold value C) has been described. In addition to the above example, the control unit 50 may determine that the knee still state occurs when the load value is equal to or below a predetermined value (an upper-limit load threshold value, a threshold value D) that is larger than the threshold value C (condition D), and may determine that the knee still state is not occurring when the load value exceeds the predetermined value (the threshold value D) and may lift the restriction on contraction of the cylinder 30. In other words, the control unit 50 may determine that the knee still state occurs when the load value is between the threshold value C and the threshold value D, and may determine that the knee still state is not occurring when the load value is not between the threshold value C and the threshold value D. In this manner, it is possible to prevent unnecessary restriction on contraction of the cylinder 30 from occurring when a user of the prosthetic leg puts a much of his/her weight on the prosthetic leg and needs to flex the knee, like when the user descends stairs. Consequently it is possible to reduce risk of falling or tumbling down the stairs when the user cannot flex the knee.

Moreover, after the contraction of the cylinder 30 is restricted, the control unit 50 may release the restriction on contraction of the cylinder 30 automatically when a rotational moment of the prosthetic knee joint 20 on the socket 11 in the extension direction exceeds a predetermined moment threshold value (threshold value E) (condition E). In this case, it is possible to prevent unnecessary restriction on contraction of the cylinder 30 when a user of the prosthetic leg descends stairs and therefore it is possible to reduce a risk of user's falling that may happen when the user is unable to flex his/her knee enough while descending the stairs. Furthermore, since the way to release the restriction on contraction of the cylinder 30 is additionally provided, the user is able to naturally lift the restriction even if the contraction of the cylinder 30 is restricted unexpectedly when the user descends stairs. Moreover when a user of the prosthetic leg tries to start walking down a slope while the contraction of the cylinder 30 is restricted, the user does not have to operate the prosthetic leg to lift the restriction on contraction of the cylinder 30 and is able to make a smooth transition from the still state to the swing phase. A rotational moment of the prosthetic knee joint 20 on the socket 11 may be calculated by the control unit 50 based on the load value provided from the load sensor 70.

In addition to the conditions A, B, and C, the control unit 50 may restrict the contraction of the cylinder 30 automatically when a user performs a set of knee flexion and extension of the above-knee prosthesis 10 one or more times for a predetermined short period of time. More specifically, the control unit 50 may automatically restrict the contraction of the cylinder 30 when the above-knee prosthesis has experienced two states within a predetermined time period (for instance, 1 to 3 seconds): one is that a knee angle value exceeds a predetermined knee angle change upper-limit threshold value (a threshold value F1) and the other is that a knee angle value falls below a predetermined knee-angle change lower-limit threshold value (a threshold value F2, here F1>F2) (condition F). In this way, even when the condition to restrict the contraction of the cylinder 30 automatically (for example, the state that satisfies all of the conditions A, B and C continues for the predetermined time duration T₁) is not satisfied, it is possible to restrict the contraction of the cylinder 30 in accordance with the intention of the user of the above-knee prosthesis. Especially, even for a user of the above-knee prosthesis who finds it difficult to stoop down slightly, flex his/her knees a little and stay still, it is possible to allow the restriction on contraction of the cylinder 30 when the user needs to do.

### Advantageous Effects of the Embodiment

As described above, according to the embodiment, the control unit 50 determines whether the knee portion 22 is in the knee still state based on a knee angular velocity value, and automatically restrict the contraction of the cylinder 30 based on an amount of time in which the knee still state continues. In this manner, it is possible to restrict contraction of the cylinder 30 in accordance with an intention of a user of the above-knee prosthesis who tries to stoop down slightly, flex his/her knees a little and stay still in this posture. Once the contraction of the cylinder 30 is restricted, the user can maintain the still state without using his/her force.

Moreover, according to the embodiment, the control unit 50 determines whether the knee portion 22 is in the knee still state based on a knee angle value in addition to the knee angular velocity. In this manner, it is possible to prevent a trouble that is caused by unnecessary restriction on contraction of the cylinder 30 when a user of the above-knee prosthesis extends his/her knees to stand upright.

Moreover, according to the embodiment, the control unit 50 determines whether the knee portion 22 is in the knee still state based on a load value in addition to the knee angular velocity value. In this manner, it is possible to prevent a trouble that is caused by unnecessary restriction on contraction of the cylinder 30 when a user of the above-knee prosthesis is seated or descends stairs.

Furthermore, according to the embodiment, it is possible to automatically restrict or lift the restriction on contraction of the cylinder 30 with the simple configuration. Among others, the above feature can be realized with a small number of sensors (for example, two sensors: the knee angle sensor 60 and the load sensor 70) so that the control becomes easier compared to the case where many sensors are used and consequently it is possible to cut a cost. Moreover, the system in the prosthetic knee joint 20 is relatively simple so that it is possible to increase the reliability.

### Modification Examples

Although the embodiments of the invention have been described above, the invention is not restricted to the above-described embodiments, and various modifications are possible within the scope of the claims. For example, the following modifications are possible.

The mechanism of the knee portion 22 described above may be a multi-bar linkage mechanism such as a four-bar linkage mechanism, in addition to a single-bar linkage mechanism.

Although the cylinder 30 has been illustrated as a hydraulic cylinder utilizing a hydraulic pressure, the cylinder 30 is not limited to this but may be any cylinder such as a pneumatic cylinder utilizing air and an electric cylinder.

In the above-described embodiment, contraction of the cylinder 30 is restricted by closing the flexion-side valve 45 by the control unit 50. However, the way to restrict the contraction of the cylinder 30 is not limited to this. Alternatively the contraction of the cylinder 30 may be restricted by physically locking movable parts of the cylinder 30 (such as the piston rod 34 and the piston 35).

In the above-described embodiment, the knee angle sensor 60 is illustrated as it includes the magnet housed in the piston rod 34 and the magnet sensor fixed to the cylinder tube 33. However the knee angle sensor 60 is not limited to this but may be the one in which the magnet sensor is housed in the piston rod 34 and the magnet is fixed to the cylinder tube 33, Alternatively the knee angle sensor 60 may be ones that directly detect a knee angle value such as a rotary encoder.

Moreover, in the above-described embodiment, the control unit 50 calculated a knee angular velocity based on a knee angle value detected by the knee angle sensor 60. However the invention is not limited to this. Instead of the knee angle sensor 60, a knee angular velocity sensor (not shown) that directly or indirectly detects the knee angular velocity value may be provided. An example of such a knee angular velocity sensor may include a gyro sensor. In this case, the control unit 50 may be coupled to the knee angular velocity sensor and determine whether the knee portion 22 is in the still state or not based on a knee angular velocity value detected by the knee angular velocity sensor. The control unit 50 may then automatically restrict the contraction of the cylinder 30 based on a time duration in which the knee portion is in the still state in the same manner as the embodiment described above.

What is claimed is:

1. A prosthetic knee joint that couples a foot portion and a socket that corresponds to a thigh of a prosthesis user, comprising:
a knee portion coupled to the socket;
a cylinder coupled to the knee portion, the cylinder limiting or aiding operation of the knee portion;
a knee angle sensor directly or indirectly detecting a knee angle value which is an angle of the knee portion; and
a control unit coupled to the knee angle sensor, the control unit controlling driving of the cylinder,
wherein the control unit calculates a knee angular velocity value based on the knee angle value detected by the knee angle sensor, and
the control unit determines whether the knee portion is in a knee still state based on the knee angular velocity value and automatically restricts contraction of the cylinder based on a time duration in which the knee portion is in the knee still state.

2. The prosthetic knee joint of claim 1, wherein
the control unit restricts contraction of the cylinder when the knee angle value is on a knee flexion side with reference to a predetermined value.

3. The prosthetic knee joint of claim 1, further comprising:
a load sensor directly or indirectly detecting a load value of the prosthetic knee joint on the foot portion, wherein
the control unit restricts contraction of the cylinder when the load value provided from the load sensor is larger than a predetermined value.

4. The prosthetic knee joint of claim 3, wherein
the control unit lifts the restriction on contraction of the cylinder when the load value exceeds an upper-limit load threshold value that is larger than the predetermined value.

5. The prosthetic knee joint of claim 1, wherein
the control unit automatically restricts contraction of the cylinder when the knee still state continues for a predetermined time duration T₁.

6. The prosthetic knee joint of claim 1, wherein
after contraction of the cylinder is restricted, the control unit automatically lifts the restriction on contraction of the cylinder when the load value is below a predetermined value or when the knee angle value shifts to a knee extension side and the knee angle value continues to be below a predetermined threshold value for a predetermined time duration T₂ or longer.

7. The prosthetic knee joint of claim 5, wherein
after contraction of the cylinder is restricted, the control unit automatically lifts the restriction on contraction of the cylinder when the load value is below a predetermined value or when the knee angle shifts to a knee extension side and the knee angle value continues to be below a predetermined threshold value for a predetermined time duration T₂ or longer, and wherein relationship between the time duration T₁ and the time duration T₂ is represented as T₁>T₂.

8. The prosthetic knee joint of claim 1, wherein
after contraction of the cylinder is restricted, the control unit automatically lifts the restriction on contraction of the cylinder when a rotational moment of the prosthetic knee joint on the socket in an extension direction exceeds a predetermined moment threshold value.

9. The prosthetic knee joint of claim 1, wherein
the control unit automatically restricts contraction of the cylinder when the prosthetic knee joint has experienced two states: one is that the knee angle value exceeds a predetermined knee angle change upper-limit threshold value and the other is that the knee angle value falls below a predetermined knee-angle change lower-limit threshold value.

10. A prosthetic knee joint that couples a foot portion and a socket that corresponds to a thigh of a prosthesis user, comprising:
a knee portion coupled to the socket;
a cylinder coupled to the knee portion, the cylinder limiting or aiding operation of the knee portion;
a knee angular velocity sensor directly or indirectly detecting a knee angular velocity value which is a change of angle of the knee portion per unit time; and
a control unit coupled to the knee angle sensor, the control unit controlling driving of the cylinder,
wherein the control unit determines whether the knee portion is in a knee still state based on the knee angular velocity value detected by the knee angular velocity sensor and automatically restricts contraction of the cylinder based on a time duration in which the knee portion is in the knee still state.

11. A prosthetic knee joint that couples a foot portion and a socket that corresponds to a thigh of a prosthesis user, comprising:
a knee portion coupled to the socket;
a cylinder coupled to the knee portion, the cylinder limiting or aiding operation of the knee portion;
a knee angle sensor directly or indirectly detecting a knee angle value which is an angle of the knee portion;
a control unit coupled to the knee angle sensor, the control unit controlling driving of the cylinder; and
a load sensor directly or indirectly detecting a load value of the prosthetic knee joint on the foot portion, wherein
after contraction of the cylinder is restricted, the control unit automatically lifts the restriction on contraction of the cylinder when the load value obtained from the load sensor is below a predetermined value or when the knee angle value shifts to a knee extension side and the shifting of the knee angle value continues for a predetermined time duration or longer.

12. A method of controlling a prosthetic knee joint that couples a foot portion and a socket that corresponds to a thigh of a prosthesis user, wherein the prosthetic knee joint includes:
a knee portion coupled to the socket;
a cylinder coupled to the knee portion, the cylinder limiting or aiding operation of the knee portion; and
a knee angle sensor directly or indirectly detecting a knee angle value which is an angle of the knee portion, the method comprising:
calculating a knee angular velocity value based on the knee angle value detected by the knee angle sensor, and
determining whether the knee portion is in a knee still state based on the knee angular velocity value and automatically restricting contraction of the cylinder based on a time duration in which the knee portion is in the knee still state.
